(19) United States
(12) Patent Application Publication
(10) Pub. No.: US 2020/0170779 A1
(43) Pub. Date: Jun. 4, 2020

(54) MEDICAL TEXTILE HAVING LOW DENIER PER FILAMENT YARN

(71) Applicant: THE SECANT GROUP, LLC, Telford, PA (US)

(72) Inventors: Mevlut TASCAN, Breinigsville, PA (US); Amanda WEBER, Macungie, PA (US); Andrew METZGER, Lafayette Hill, PA (US)

(21) Appl. No.: 16/697,916

(22) Filed: Nov. 27, 2019

Related U.S. Application Data
(60) Provisional application No. 62/773,669, filed on Nov. 30, 2018.

(51) Int. Cl. A61F 2/07; D03D 15/00

(52) U.S. Cl. CPC A61F 2/07; D03D 15/0061; A61F 2240/001; D10B 2331/04; D10B 2509/06

(57) ABSTRACT

An engineered textile including a low dpf yarn and medical applications including the low dpf yarn. The denier per filament of the low dpf yarn is less than 0.50 and the water permeability of the engineered textile is less than 500 mL/min/cm².

### RELATED APPLICATIONS

This application claims the benefit of, and priority to, U.S. App. No. 62/773,669 filed Nov. 30, 2018, which is hereby incorporated by reference in its entirety.

### FIELD

The present invention is directed to woven fabrics formed from low denier per filament yarns. In particular, the present invention is directed to implantable medical textiles having low water permeability, improved tissue infiltration, surface smoothness, and adhesion, and decreased thickness.

### BACKGROUND

In medical devices such as artificial heart valves and endovascular grafts there is a need for water impermeable fabric to act as a conduit for blood transfer and a barrier between the blood and native tissue.

Medical textile tubes have been used in endovascular aneurysm repair (EVAR) procedures to create a conduit for blood flow when intervention is needed. The woven construct, sometimes referred to as a stent-graft, creates a barrier to block blood flow to aneurysms while also acting as a conduit for blood flowing through the endovascular regions. See, for example, U.S. Patent Application Publication No. 2002/052649, entitled “Graft having region for biological seal formation,” by Greenhalgh, published May 2, 2002 and “Woven stent/graft structure,” by Greenhalgh, U.S. Pat. No. 6,159,239. Most medical procedures use a wire/metal scaffold sewn around the textile tube, which acts to support the weak area that needs repair.

In minimally invasive surgeries the implanted devices are delivered through a catheter-based delivery system. The fabric needs to be thin and flexible enough to be compressed in the catheter, while also having low water permeability and good suture strength. For example, in endovascular aneurysm repair (EVAR) procedures the textile occupies up to 30% of the space within the delivery device.

### SUMMARY

In an embodiment, an engineered textile includes a low dpf yarn. The denier per filament of the low dpf yarn is less than 0.50 and the water permeability of the engineered textile is less than 500 mL/min/cm².

### BRIEF DESCRIPTION OF THE DRAWINGS

FIG. 1 is an electron microscope image of an engineered textile formed from low dpf filament yarns, according to an embodiment.

FIG. 2 is an electron microscope image of an engineered textile formed from low dpf filament yarns, according to an embodiment.

FIG. 3 is an electron microscope image of a comparative conventional engineered textile.

FIG. 4 is an electron microscope image of an engineered textile formed from low dpf filament yarns, according to an embodiment.

FIG. 5 is an electron microscope image of an engineered textile formed from low dpf filament yarns, according to an embodiment.

FIG. 6 is an electron microscope image of a comparative conventional engineered textile.

FIG. 7 is a graph of the water permeability by average pore size of engineered textiles formed from low dpf filament yarns and comparative conventional engineered textiles.

FIG. 8 is a schematic representation of a bifurcated graft in accordance with an embodiment.

Wherever possible, the same reference numbers will be used throughout the drawings to represent the same parts.

### DETAILED DESCRIPTION

To address these shortcomings in the art, provided are woven, braided or knit engineered textiles for use in implantable medical devices as a high surface area substrate to increase infiltration during cellular expansion. Contour guidance is the natural propensity for growing tissue cells to follow the contour features of a surface as the tissue expands. In certain medical applications, such as heart surgery (e.g., stent implantation, heart valve replacement or repair), the tissue expands to colonize the textile, as part of the healing process. The colonization into a textile structure, such as a stent, is a form of bonding mechanism of textile to tissue. Engineered textiles of the present invention incorporate low denier per filament multi-filament yarns in at least one of the warp or weft of the engineered textile, resulting in increased surface area and small pore size. Implantable medical devices formed from the textiles exhibit improved tissue colonization of the implanted textile. In one embodiment, a woven textile is formed using 20 denier 68 filament Low dpf polyester. In comparison to 20 denier 18 filament PET constructions of similar picks per inch, ends per inch, and weave pattern, the 20/68 Low dpf PET outperformed the comparative by having small pores and lower water permeability.

The engineered textiles incorporate structural or coating properties that lead to improved medical outcomes compared to current commercial products. These can include modification of physiochemical properties such as water permeability, smoothness, coating stiffness, porosity, and surface chemistry of the textile coating. Additionally, exemplary embodiments can still deliver appropriate suture strength, such as in excess of 8N.

To provide a fluid-tight textile, fabrics in accordance with exemplary embodiments comprise approximately 100-450 ends per inch (“EPI”) at approximately 75-200 picks per inch (“PPI”), typically at least 150 ends per inch. In some embodiments, the fabric may comprise approximately 325-400 EPI at 100-175 PPI. In other embodiments, the fabric comprises about 150 EPI at 100 PPI. In still other embodiments, the textile may comprise approximately 165to 300 EPI at 100-175 PPI. In some embodiments, the textile is a flat textile. In some embodiments, the textile is manufactured as a flat woven tube comprising two faces.

The textile may be formed from various woven, knit, or braided constructions, including but not limited to a double needle bar knit, tricot warp knit, a plain weave, twill weave, rib weave (e.g., warp rib or weft rib), satin weave, sateen weave, mock leno weave, and/or herringbone weave. In some embodiments, the textile is formed from a plain weave, a twill weave, weft rib, or satin weave. In one embodiment, the textile is formed from a plain weave. In one embodiment, the textile is formed from a 2/2 twill weave.

In an embodiment, the woven, braided, or knit construction may include filaments, fibers, or yarns having differing fiber cross-sections. In some embodiments, the cross sections may include circular, elliptical, multi-lobal (e.g., bilobal, trilobal, tetralobal), triangular, lima bean, lobular, flat, and/or dog-bone cross-sections. In some embodiments, the fibers and/or yarns may be single or multifilament fibers, or yarns. In some embodiments, the construction may include fibers having islands-in-the-sea type cross-sections. The engineered textile may be a single or multi-layered textile.

In some embodiments, the braided, knit, or woven textile includes a multifilament yarn having a yarn denier of at least 5 denier, at least 7 denier, at least 10 denier, at least 12 denier, at least 15 denier, at least 18 denier, at least 20 denier, less than 50 denier, less than 45 denier, less than 35 denier, less than 30 denier, less than 25 denier, less than 23 denier, less than 21 denier, and ranges and subranges thereof In some embodiments, the braided, knit, or woven textile consists of multi-filament yarns having a yarn denier of at least 5 denier, at least 7 denier, at least 10 denier, at least 12 denier, at least 15 denier, at least 18 denier, at least 20 denier, less than 30 denier, less than 25 denier, less than 23 denier, less than 21 denier, and ranges and subranges thereof.

In an embodiment, the braided, knit, or woven textile may include a plurality of fibers or yarns having differing number of fibers and yarn deniers (den). In some embodiments, the yarn deniers may be at least 10 den, at least 12 den, at least 15 den, at least 17 den, at least 20 den, less than 200 den, less than 150 den, less than 120 den, less than 100 den, less than 80 den, less than 60 den, less than 40 den, less than 35 den, less than 33 den, less than 30 den, less than 28 den, less than 25 den, less than 23 den, less than 21 den, and ranges and subranges thereof.

In some embodiments, the yarns of the braided, knit, or woven textile include a multi-filament yarn having an average denier per filament (dpf) of less than 0.50 dpf, less than 0.40 dpf, less than 0.35 dpf, less than 0.33 dpf, less than 0.30 dpf, less than 0.28 dpf, less than 0.26 dpf, less than 0.24 dpf, greater than 0.10 dpf, greater than 0.12 dpf, greater than 0.15 dpf, greater than 0.18 dpf, greater than 0.20 dpf, greater than 0.22 dpf, and ranges and subranges thereof In some embodiments, the yarns of the braided, knit, or woven textile consist of filaments having an average denier per filament (dpf) of less than 0.50 dpf, less than 0.40 dpf, less than 0.35 dpf, less than 0.33 dpf, less than 0.30 dpf, less than 0.28 dpf, less than 0.26 dpf, less than 0.24 dpf, greater than 0.10 dpf, greater than 0.12 dpf, greater than 0.15 dpf, greater than 0.18 dpf, greater than 0.20 dpf, greater than 0.22 dpf, and ranges and subranges thereof. In one embodiment, the braided, knit, or woven textile includes a yarn that consists of filaments of less than 0.30 dpf. In one embodiment, the braided, knit, or woven textile consists of yarns further consisting of filaments of less than 0.30 dpf.

In some embodiments, the braided, knit, or woven textile includes a multi-filament yarn in which the filaments have an average cross-section of less than 8.0 micrometers, less than 6.0 micrometers, less than 5.5 micrometers, less than 5.0 micrometers, less than 4.8 micrometers, less than 4.5 micrometers, at least about 2.0 micrometers, at least about, 3.0 micrometers, at least about 3.5 micrometers, at least about 4.0 micrometers, and ranges and subranges thereof. In some embodiments, the braided, knit, or woven textile consists of one or more multi-filament yarns in which the filaments have an average cross-section of less than 6.0 micrometers, less than 5.5 micrometers, less than 5.0 micrometers, less than 4.8 micrometers, less than 4.5 micrometers, at least about 2.0 micrometers, at least about, 3.0 micrometers, at least about 3.5 micrometers, at least about 4.0 micrometers, and ranges and subranges thereof.

The braided, woven, or knit textile may exhibit a uniform or non-uniform thickness. In some embodiments, the textile thickness is substantially uniform across the face of the textile. In some embodiments, the thickness of textile may be at least 25 micrometers, at least 30 micrometers, at least 35 micrometers, at least 40 micrometers, at least 42 micrometers, at least 45 micrometers, at least 50 micrometers, at least 60 micrometers, about 61 micrometers, less than 100 micrometers, less than 90 micrometers, less than 80 micrometers, less than 70 micrometers, less than 65 micrometers, less than 62 micrometers, and ranges and subranges thereof.

The braided, woven, or knit textile may be formed from any resorbable material, non-resorbable material, or combination of materials suitable for weaving. Suitable non-resorbable materials include, but are not limited to, polyethylene terephthalate (PET), polypropylene (PP), poly(vinylidene fluoride) (PVDF), silicone, polyurethane, polycarbonate, polyether ketone, collagen, fibronectin, hyaluronic acid, and combinations thereof. Suitable resorbable materials include, but are not limited to, polycaprolactone (PCL), polylactic acid (PLA), polyglycolic acid (PGA), poly(lactic-co-glycolic acid) (PLGA), poly(glycerol sebacate) (PGS), Lysine-poly(glycerol sebacate) (KPGS), collagen, fibrin, alginate, silk, and combinations thereof In some embodiments, the scaffold may include polyethylene terephthalate (PET). In one embodiment, the textile may be formed from polyethylene terephthalate (PET). In one embodiment, the textile includes a PET fiber having a round profile.

In some embodiments, a coating may be provided to the fibers or yarns of the textile. In some embodiments, the coating may be applied to the fibers or yarns prior to the formation of the textile. In some embodiments, the coating may be applied after formation of the textile structure. In some embodiments, the coating may be formed from resorbable materials. The resorbable materials may enhance endogenous regeneration of tissue. Suitable resorbable materials include, but are not limited to, polycaprolactone (PCL), polylactic acid (PLA), polyglycolic acid (PGA), poly(lactic-co-glycolic acid) (PLGA), poly(glycerol sebacate) (PGS), Lysine-poly(glycerol sebacate) (KPGS), poly(glycerol sebacate urethane) (PGSU), amino-acid incorporated PGS, and combinations thereof In some embodiments, the coatings may be applied by spray or dip coating, or lamination. The coating may improve cellular attachment to the textile.

In certain medical applications, such as heart valve replacement or repair, it may be desirable to have a water impermeable barrier. In some embodiments, the water permeability of the textile prior to any coatings is less than 500 mL/min/cm², less than 400 mL/min/cm², less than 375 mL/min/cm², less than 350 mL/min/cm², less than 325 mL/min/cm², less than 300 mL/min/cm², less than 275 mL/min/cm², less than 250 mL/min/cm², less than 225 mL/min/cm², less than 200 mL/min/cm², less than 150 mL/min/cm², less than 100 mL/min/cm², less than 75 mL/min/cm², less than 50 mL/min/cm², less than 30 mL/min/cm², less than 20 mL/min/cm², less than 10 mL/min/cm², less than 5 mL/min/cm², less than 3 mL/min/cm², and/or less than 1 mL/min/cm².

By coating the textile with a bioresorbable or non-bioresorbable materials, the water permeability can be further reduced. Suitable non-bioresorbable materials include polyurethanes (PU). Suitable bioresorbable polymers include the bioresorbable materials described above. In some embodiments, the bioresorbable material may also encourage endogenous regeneration of tissue. Alternatively, in some embodiments, the textile may be calendared to densify the textile and reduce the water permeability. In some embodiments, the textile may be both calendared and coated.

In some embodiments, using low dpf yarn in both the warp and the weft directions in combination with mechanical alteration such as calendaring can result in a textile having a water permeability less than 6 mL/min/cm² without the use of a coating.

The choice of additional features, such as weave structure or yarn profile, may be based on patient condition, age and type of implant involved to optimize colonization. For instance, a patient may be at different stages of regenerative proliferation whereby some topography may be amenable to limited colonization capability. Topography may be used to optimize cellular colonization to improve medical outcomes.

In some embodiments, the textile may be a seamless conduit formed as a flat woven tubular textile. The weave may be any of a variety of weaves, including, but not limited to plain, basket and twill weaves. In some embodiments, the textile is formed of a plain double cloth weave forming a flattened tubular structure. The characteristics of the weave pattern may vary depending upon the application for the textile. However, in one embodiment, the textile is formed so that the walls are substantially impermeable to fluid, so that the graft forms a lumen that is substantially fluid-tight along its length and includes an inlet and an outlet. For example, when used in a vascular application, the walls of the graft are substantially impermeable to blood so that the graft forms a conduit permitting the flow of blood along the axis of the tubular textile while impeding blood leakage through the sidewalls of the graft.

The method for producing the textile will be described. In some embodiments, the textile is woven on a loom configured to produce a plain weave double cloth textile. The loom may be any of a variety of types, including, but not limited to a jacquard loom, a circular loom or a dobby loom. In one embodiment, the textile is produced on a dobby loom.

In exemplary embodiments, the entire graft is coated with a bioresorbable material in order to minimize inflammation and encourage tissue regeneration.

The fabric may be cleaned and then heat set. In one embodiment, the fabric is heat set at about 205° C. for dimensional stability. In one embodiment, the fabric is calendared at a temperature of about 149° C. (300° F.).

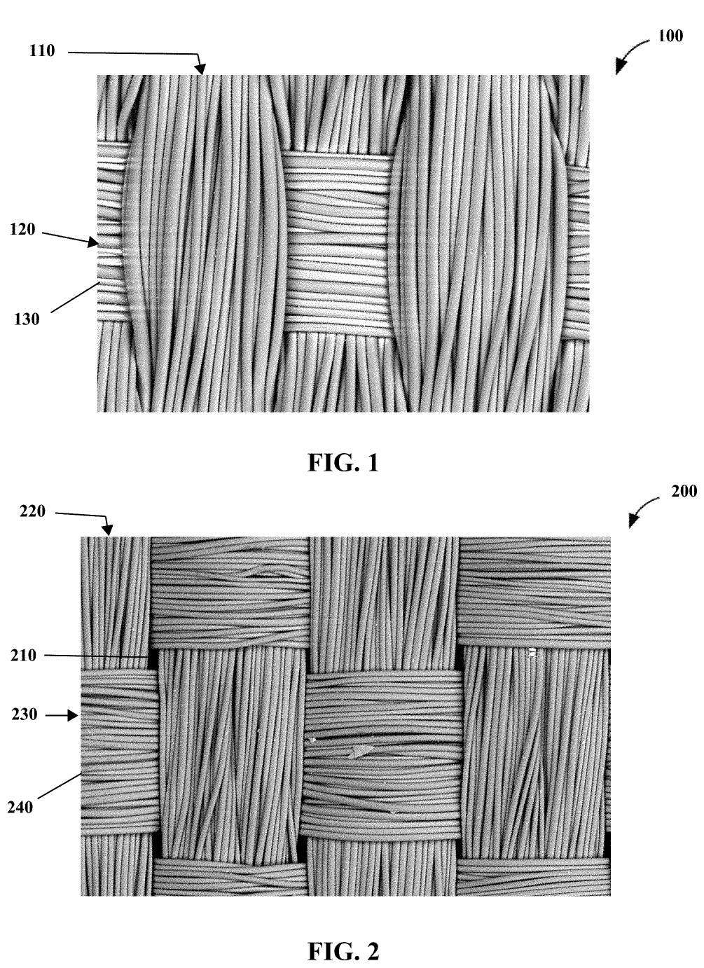

FIG. 1 provides an example embodiment of an engineered textile 100 formed from low denier per filament (dpf) yarns. In the embodiment of FIG. 1, the textile is a plain weave that has polyethylene terephthalate (PET) yarns in both the warp 110 and weft 120 of the fabric. The PET yarns are approximately 20 denier (den) yarns having 68 filaments 130 (20/68). The PET filaments have a substantially circular cross-section and an average diameter of about 5 micrometers.

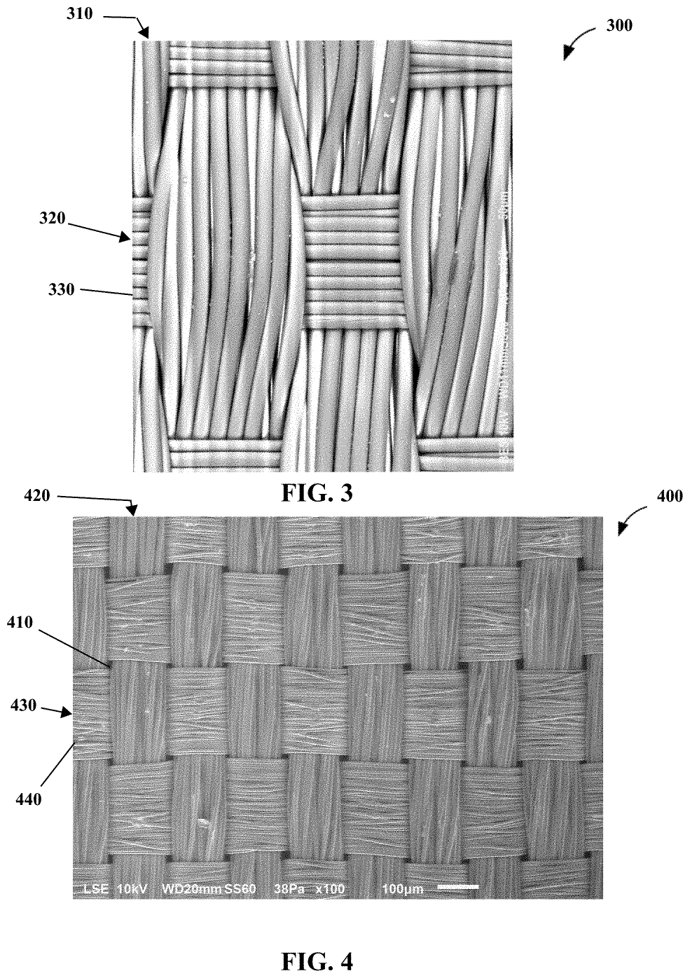

FIG. 2 provides an example embodiment of an engineered textile 200 formed from low denier per filament (dpf) yarns. In the embodiment of FIG. 2, the textile illustrates micropores 210. The textile is a weft rib having polyethylene terephthalate (PET) yarns in both the warp 220 and weft 230 of the fabric. The PET yarns are approximately 20 denier (den) yarns having 68 filaments 240 (20/68). The PET filaments have a substantially circular cross-section and an average diameter of about 5 micrometers. The measured pore x (cross machine direction) size ranges between 6 and 10 micrometers. The measured pore y (machine direction) size ranges between 24 and 29 micrometers.

FIG. 3 provides a comparative example of an engineered textile 300 formed from PET yarns. In the comparative example of FIG. 3, the textile is a plain weave that has polyethylene terephthalate (PET) yarns in both the warp 310 and weft 320 of the fabric. The PET yarns are approximately 20 denier (den) yarns having 18 filaments 330 (20/18). The PET filaments have a substantially circular cross-section and an average diameter of greater than 10 micrometers.

FIG. 4 provides an example embodiment of an engineered textile 400 formed from low denier per filament (dpf) weft yarns. In the embodiment of FIG. 4, the textile illustrates micropores 410. The textile is a weft rib weave having polyethylene terephthalate (PET) yarns in both the warp 420 and weft 430 of the fabric. The PET weft yarns are approximately 20 denier (den) yarns having 68 filaments 440 (20/68). The PET weft filaments have a substantially circular cross-section and an average diameter of about 5 micrometers. The PET warp yarns are approximately 20 denier (den) yarns having 18 filaments 440 (20/18). The PET warp filaments have a substantially circular cross-section and an average diameter of greater than 10 micrometers. The measured pore x (cross machine direction) size ranges between10 and 40 micrometers. The measured pore y (machine direction) size ranges between 10 and 50 micrometers.

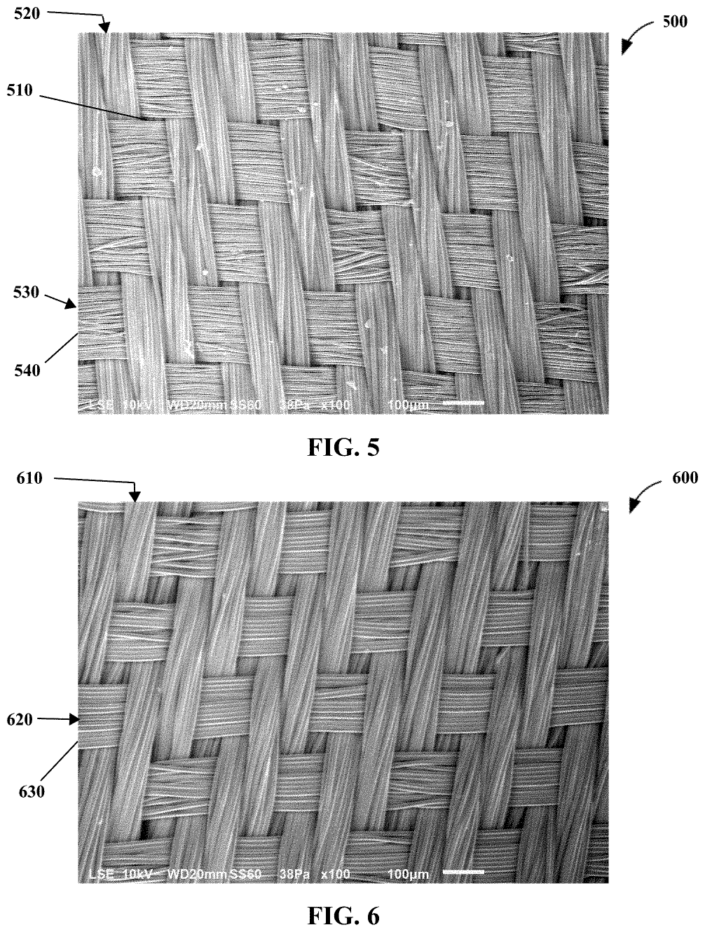

FIG. 5 provides an example embodiment of an engineered textile 500 formed from low denier per filament (dpf) weft yarns. In the embodiment of FIG. 5, the textile illustrates micropores 510. The textile is a twill weave having polyethylene terephthalate (PET) yarns in both the warp 520 and weft 530 of the fabric. The PET weft yarns are approximately 20 denier (den) yarns having 68 filaments 540 (20/68). The PET weft filaments have a substantially circular cross-section and an average diameter of about 5 micrometers. The PET warp yarns are approximately 20 denier (den) yarns having 18 filaments 540 (20/18). The PET warp filaments have a substantially circular cross-section and an average diameter of greater than 10 micrometers.

FIG. 6 provides a comparative example of an engineered textile 600 formed from (PET) yarns. In the comparative example of FIG. 6, the textile is a twill weave that has polyethylene terephthalate (PET) yarns in both the warp 610 and weft 620 of the fabric. The PET yarns are approximately 20 denier (den) yarns having 18 filaments 630 (20/18). The PET filaments have a substantially circular cross-section and an average diameter of greater than 10 micrometers.

### EXAMPLES

TABLE 1

| | Inventive | Inventive | Comparative |
|---|---|---|---|
| | Example 1 | Example 2 | Example 1 |
| | (FIG. 1) | (FIG. 2) | (FIG. 3) |
| | Warp: | Warp: | Warp: |
| | PET (20/68) | PET (20/68) | PET (20/18) |
| | Weft: | Weft: | Weft: |
| | PET (20/68) | PET (20/68) | PET (20/18) |
| Thickness | 88 micrometers | 53 micrometers | 81 micrometers |
| EPI | 291 | 326 | 306 |
| PPI | 141 | 138 | 142 |
| Water Permeability | 83 | 6 | 364 |
| (mL/min/cm²) | | | |

TABLE 2
Weft rib construction

| | Inventive | Comparative |
|---|---|---|
| | Example 3 | Example 2 |
| | (FIG. 4) | (FIG. 6) |
| | Warp: PET (20/18) | Warp: PET (20/18) |
| | Weft: PET (20/68) | Weft: PET (20/18) |
| Thickness | 70 micrometers | 73 micrometers |
| EPI | 361 | 347 |
| PPI | 95 | 103 |
| Water Permeability | 379 | 882 |
| (mL/min/cm²) | | |

In the example of Table 3 below, a 2×2 twill weave was prepared using (20/18) PET yarn in the warp and (20/68) PET yarn in the weft.

TABLE 3
2x2 Twill weave construction (Inventive Example 4) (FIG. 5)

| | | | | Pore x | Pore y | Water |
|---|---|---|---|---|---|---|
| | | | Thickness | direction | direction | permeability |
| Specimen | EPI | PPI | (micrometers) | (micrometers) | (micrometers) | (mL/min/cm²) |
| 1 | 336 | 122 | 72 | 32 | 25 | 275 |
| 2 | 336 | 124 | 73 | 30 | 38 | 289 |
| 3 | 344 | 122 | 72 | 31 | 48 | 373 |
| Avg | 339 | 123 | 72 | 31 | 37 | 312 |

In the (comparative) example of Table 4 below, a 2×2 twill weave was prepared using (20/18) PET yarn in the warp and (20/18) PET yarn in the weft.

TABLE 4
2x2 Twill weave construction (Comparative)(FIG. 6)

| | | | | Pore x | Pore y | Water |
|---|---|---|---|---|---|---|
| | | | Thickness | direction | direction | permeability |
| Specimen | EPI | PPI | (micrometers) | (micrometers) | (micrometers) | (mL/min/cm²) |
| 1 | 344 | 128 | 84 | 56 | 103 | 1156 |
| 2 | 336 | 126 | 82 | 53 | 76 | 1186 |
| 3 | 344 | 126 | 84 | 44 | 84 | 912 |
| Avg | 341 | 127 | 83 | 51 | 88 | 1085 |

Surface area has a significant role in the performance of fabrics created with low dpf yarn. When comparing 20 den yarn with 18 filaments that has a typical filament diameter of 10 micrometers to 20 den with 68 filaments yarn of 5 micrometer filament diameter there is a 1.9× increase in lateral surface area. When comparing a standard 40 denier with 27 filaments to a Low Denier Per Filament yarn such as 40 den (2/20/68) with 136 filaments there is a 2.5× increase in lateral surface area.

By replacing the weft yarn element in the woven fabric with a low denier per filament yarn a large decrease in water permeability is observed. For example, a 20 den yarn with 68 filaments per bundle replaced a 20 den 18 filament yarn in the weft region and reduced the water permeability by 43% with the same fabric density.

Low dpf yarns otherwise known as microdenier yarns have the ability to reduce porosity which may be a key feature to reducing water permeability for medical implantable textiles. Since the filaments are so small, they are able to lay much flatter than a yarn with higher diameter filaments which also will also give the added benefit of making a smoother and thinner fabric. In medical device applications, when the device is being deployed the smoother and thinner fabric allows the device to easily slide out of the delivery system and into the body with reduced abrasion. Additionally, it was surprisingly discovered that even when porosity was not decreased, exemplary embodiments still displayed reduced water permeability over conventional textiles having a similar pore size. Exemplary embodiments allow for lower density even at the same porosity due to the way the filaments splay out in the low dpf yarns. When used as the same density as a comparable fabric made entirely of 20/18 yarns, the use of low dpf yarn results in lower porosities. Thus, even at similar porosities exemplary embodiments are providing lower water permeability, which is believed to be a product of the yarn to yarn friction.

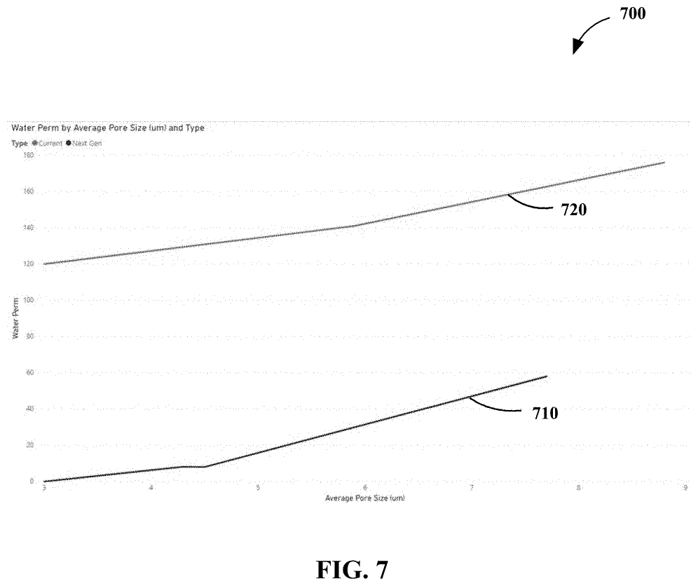

FIG. 7 graphically presents the effects of pore size and surface area on the water permeability of the engineered textile 700. In FIG. 7, the water permeability of engineered textiles formed from low dpf yarns as a function of average pore size is shown as element 710. The water permeability of comparative conventional engineered textiles having similar areal density and formed from conventional yarns as a function of average pore size is shown as element 720.

In the example of FIG. 7, it is believed that during the water permeability test the starting pore sizes increase significantly under the hydrostatic pressure of the test. It is further believed that that the increased number of filaments of the low dpf fiber increases the fiber to fiber interactions and reduces or prevents the fabric pores from opening under pressure, thus resulting in the low water permeability results. In some of the fabrics tested the low dpf fabric has lower end and pick density than the conventional engineered textile.

As demonstrated, engineered textiles formed from low dpf yarns exhibit substantially reduced water permeability. The weave structure plays an important role in how smooth the surface of a textile is. Generally, the longer the yarn floats the smoother the fabric becomes and the more interlacements it has can lead to a rougher surface. For example, a satin structure is typically smoother than a plain weave structure. By using low dpf yarn the smoothness is even further enhanced by increasing the surface area.

Low dpf yarns additionally benefit the fabric by creating smaller spaces in which smaller sized cells can infiltrate and proliferate. The colonization of these cells within the pockets of the low dpf yarn will potentially allow better joining of the native tissue to the implantable textile.

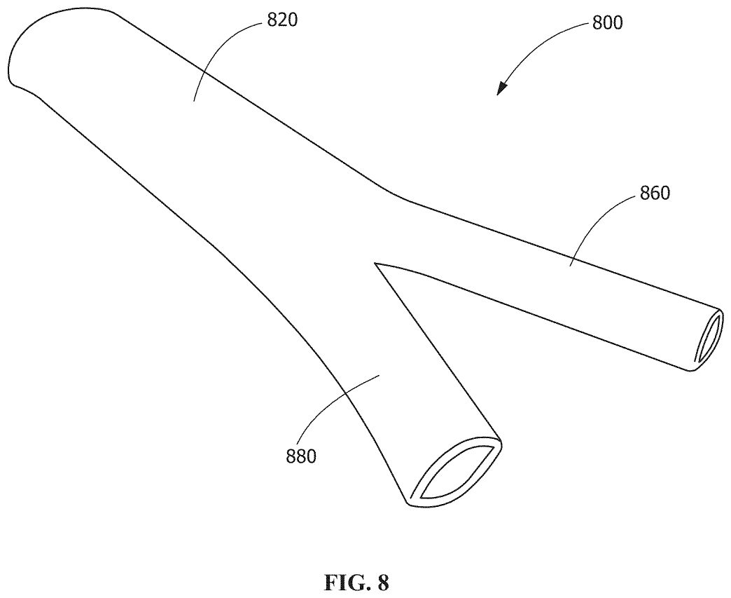

The engineered textiles may be used in a variety of medical and other applications for both broad cloth and lumen implantables and may be particularly advantageous for use in forming grafts, valves and other articles, include vascular grafts and heart valve prosthetic devices. FIG. 8 illustrates a bifurcated lumen 800 including an engineered textile. Lumens and other implantable articles may be formed, for example, as a woven tube or as a flat cloth which may be appended to a support or frame.

As illustrated in FIG. 8, the bifurcated lumen 800 includes a main body portion 820 having a bifurcated portion 840 extending therefrom. The main body portion 820 forms a single lumen including one or more engineered textiles that transitions into two separate lumens 860, 880 at the bifurcated portion 840. As will be appreciated by those skilled in the art, although shown as including both the main body portion 820 and the bifurcated portion 840, lumen 800 may include any individual section or portion thereof and may be further furcated depending upon the application. The main body portion 820 and the separate lumens 860, 880 formed by the bifurcated portion 840 each include any suitable size, shape, and/or orientation.

Exemplary embodiments include a woven textile formed using 20 denier 68 filament Low DPF polyester. In comparison to 20 denier 18 filament PET constructions of similar picks per inch, ends per inch, and weave pattern, the 20/68 Low DPF PET outperformed its predecessor by having small pores and lower water permeability. Additionally exemplary embodiments exhibit high suture tensile strengths, with suture retention tests in excess of 8N, such as 10 N or greater for textiles made with 20 denier yarns. Surprisingly, suture retention increases for exemplary embodiments even with fewer EPI than conventional fabrics.

Low DPF yarn is not available commercially for medical grade applications and due to the sizes of openings that surround the filaments it is perfectly suitable for endothelial cell growth.

Exemplary embodiments may be used, for example, in any vascular graft, heart valve prosthetic device and hollow lumen organ requiring a textile for adhesion and water impermeability.

Low DPF yarn in knitted constructions can be used to increase cellular growth in the open space around the microdenier filaments.

This approach can also be utilized in braiding to increase surface coverage while decreasing density or picks per inch.

Other applications for exemplary embodiments also include flexible sutures that have lower suture drag due to smoothness of Low DPF yarn, cardiovascular patches where water permeability and flexibility are essential for successful surgeries, wound care applications where increasing surface area is needed but not bulk, and embolic protection devices that need a thin, smooth textile for delivery systems.

While the invention has been described with reference to one or more embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. In addition, all numerical values identified in the detailed description shall be interpreted as though the precise and approximate values are both expressly identified.

What is claimed is:

1. An engineered textile comprising a low dpf yarn, wherein the denier per filament of the low dpf yarn is less than 0.50 and wherein the water permeability of the engineered textile is less than 500 mL/min/cm².

2. The engineered textile of claim 1, wherein the low dpf yarn is a polyethylene terephthalate (PET) yarn.

3. The engineered textile of claim 1, wherein the denier of the low dpf yarn is between 15 and 25.

4. The engineered textile of claim 1, wherein the water permeability of the engineered textile is less than 300 mL/min/cm².

5. The engineered textile of claim 1, wherein engineered textile comprises in the range of 100 to 450 ends per inch and in the range of 75 to 200 picks per inch.

6. The engineered textile of claim 5, wherein engineered textile has in the range of 325 to 400 ends per inch and in the range of 100 to 175 picks per inch.

7. The engineered textile of claim 5, wherein engineered textile has about 150 ends per inch and about 100 picks per inch.

8. The engineered textile of claim 1, wherein the engineered textile is a woven textile and a weft of the engineered textile includes the low dpf yarn.

9. The engineered textile of claim 8, wherein the engineered textile is a woven textile and the warp and the weft of the engineered textile include the low dpf yarn.

10. The engineered textile of claim 8, wherein the engineered textile includes a 20 denier 68 filament PET yarn.

11. The engineered textile of claim 1, wherein the engineered textile is a plain weave, a twill weave, a 2/2 twill weave, a weft rib weave, or a satin weave.

12. The engineered textile of claim 1 having a thickness between 25 micrometers and 100 micrometers.

13. The engineered textile of claim 1, wherein the textile has a water permeability less than 500 mL/min/cm² in an uncoated state.

14. The engineered textile of claim 1, wherein the textile is a flat cloth or a woven tube.

15. A method of forming an engineered textile comprising providing a low dpf yarn, wherein the denier per filament of the low dpf yarn is less than 0.50; and
weaving the engineered textile from the low dpf yarn to form the engineered textile having a water permeability less than 500 mL/min/cm².

16. The method of claim 15 comprising weaving using the low dpf yarn as the weft yarn.

17. The method of claim 15 whether the low dpf yarn comprises a 20 denier yarn having greater than 40 filaments.

18. The method of claim 15 further comprising calendaring the engineered textile after weaving.

19. An implantable medical device comprising the engineered textile of claim 1.

20. The implantable medical device of claim 19, wherein the implantable medical device is a vascular graft or a heart valve prosthetic device.
